(19) United States
(12) Patent Application Publication
(10) Pub. No.: US 2021/0022324 A1
(43) Pub. Date: Jan. 28, 2021

(54) EMBRYONIC ZEBRAFISH MODELS USING DNAZYME MEDIATED KNOCKDOWN

(71) Applicant: DR. REDDY'S INSTITUTE OF LIFE SCIENCES, Hyderabad (IN)

(72) Inventors: Aarti SEVILIMEDU, Hyderabad (IN); Pushkar KULKARNI, Hyderabad (IN)

(21) Appl. No.: 16/978,537

(22) PCT Filed: Feb. 15, 2019
(86) PCT No.: PCT/IB19/51255

§ 371 Date: Sep. 4, 2020

(30) Foreign Application Priority Data
Mar. 5, 2018 (IN) 201741031477

(51) Int. Cl. A01K 67/027; C12N 15/113

(52) U.S. Cl. CPC A01K 67/0276; C12N 15/113; C12N 2310/127; A01K 2227/40; A01K 2267/0306; A01K 2217/075

(57) ABSTRACT

The present invention provides DNAzymes for reduction in the expression of the target gene implicated in disease. The present invention also provides a platform technology for the use of DNAzymes to create a reduction in gene expression in zebrafish embryos. More particularly, the present invention describes DNAzyme mediated knockdown of Fmr1 mRNA, gcdh mRNA and gba1mRNA in zebrafish embryos. The present invention also provides a method of creating a model of Fragile X syndrome in Zebrafish embryos.

### RELATED APPLICATION

This application is related to Indian Complete Application 201741031477 filed on Mar. 5, 2018 and is incorporated herein in its entirety.

### FIELD OF THE INVENTION

The present invention relates to DNAzymes for reduction in the expression of the target gene implicated in a disease, in zebrafish embryos. The present invention also relates to the method of using DNAzymes to create a knockdown in zebrafish embryos. More particularly, the present invention relates to DNAzyme mediated knockdown of fmr1 mRNA, gcdh mRNA and gba1mRNA. The present invention also relates to a method of creating a model of Fragile X syndrome, Glutaricaciduria Type I and Gaucher's Disease in Zebrafish embryos.

### BACKGROUND OF THE INVENTION

Zebrafish are described as “canonical vertebrates” because of the similarities they have with mammalian biology (Curr Opin Genet Dev 10(3):252). There is a high degree of homology between the zebrafish and mammalian proteome (82% similarity) which makes functional genetic studies possible. Zebrafish has been used to model a wide range of diseases including mitochondrial diseases (Transl Res 163(2):79), genetic kidney diseases, diseases of the brain (Eur J Neurosci 42(2):1746), heart (J Cardiovasc Dev Dis 3(2):13), liver and muscle (muscular dystrophies) (Dis Model Mech 5(6):726) as well as various cancers (Curr Pathobiol Rep 2(2):61). In addition to their general utility as a model organism, Zebrafish is especially powerful for the study of normal and diseased states during early development, since the embryo is accessible for manipulation and observation from 1-cell stage. This makes zebrafish ideal for the creation of newborn and pediatric diseases with a genetic basis.

Transient as well as stable genetic alterations are possible in Zebrafish and a wide variety of tools are available to achieve the same. Gene deletions and insertions have been made predominantly using the Toll transposon excision system (Nucleic Acids Res 41(2):e36-e36), site specific alterations using the Zinc finger nucleases (ZFNs) and TALENS (Genomics 39(9):421), and more recently genome editing has been carried out using CRISPR technology (Biotechnol 31(3):227). Transient knockdown of gene expression is achieved through the use of Morpholino oligonucleotides, however more recently, a number of artifacts and off-target effects have been reported with their use (Dev Cell 35(2):145). Other methods include the use of Antisense oligonucleotides, which bind to and cleave the RNA in an RNaseH dependent fashion (PLoS One 10(10):e0139504). RNA interference is not popular in the Zebrafish community, presumably because of prevalent genome duplication leading to less efficacious RNAi mediated knockdown (Brief Funct Genomics 10(4):189-196).

One more approach for transient in vivo manipulation of gene expression relies on the use of catalytic DNA oligonucleotides (catDNAs or DNAzymes). An RNA cleaving DNAzyme is a catalytically active DNA oligo, with the ability to cleave a target RNA molecule at a specific site. The specificity arises from the complementarity of part of the oligo to the target RNA sequence (Chem Biol 1(4):223). The power of the DNAzyme approach lies in the fact that the DNAzyme contains both specificity and catalytic activity in one simple commercially synthesized oligo and that is all that is needed to create a robust knockdown. DNAzymes have been shown to cleave their target effectively in vitro, in cells, serum, in model organisms like mice and in humans (clinical trials) (Trends Biochem Sci 41(7):595; Anal Chem 87(7):4001). In studies in zebrafish, rodents and man, DNAzymes have been found to be safe and well tolerated as well.

The present invention provides a methodology to create a knockdown of a specific mRNA in zebrafish embryos using specific DNAzymes. It involves the design of specific, potent, novel DNAzymes and delivery of the DNAzymes into 0-6 hpf (hours post fertilization) zebrafish embryos using electroporation, microinjection or other delivery protocols (including but not limited to transfection, receptor mediated transport, other active or passive delivery) in a high throughput manner to generate a significant reduction in the target mRNA in the treated embryos. The present invention thus provides a quick, robust, inexpensive and high throughput methodology to create knockdowns in zebrafish, to generate disease models for various genetic diseases. An exemplary methodology is demonstrated using Fragile X syndrome where the novel DNAzyme is delivered via electroporation or microinjection. Furthermore, the invention is also supported by experimental evidence for DNAzyme based knockdown and model creation for two other monogenic diseases namely Gaucher's disease and Glutaric Aciduria Type 1.

Fragile X syndrome (FXS) is the leading cause of intellectual disabilities in males and a major monogenic cause of ASD (Autism spectrum disorders). FXS is caused due to the loss of FMR protein (FMRP).

Gaucher's disease (GD) is the most common lysosomal storage disorder (LSD), and is caused due to the mutations in the gba1 gene (Am J Hum Genet 2000; 66:1777). Most mutations result in a loss of function of the glucocerebrosidase enzyme encoded by gba1, which leads to the accumulation of its substrates glucocerebroside and glucosylsphingosine (Lancet 2008; 372:1263). Accumulation of these metabolites especially in macrophages is a characteristic feature of this disease.

Glutaricaciduria type I (GA1) is an autosomal recessive IEM disorder that arises due to mutations in the gene that encodes the enzyme Glutaryl Co-A dehydrogenase (GCDH) essential for the catabolism of lysine, hydroxylysine and tryptophan. With reduced or absent GCDH, the metabolites GA (glutaric acid) and 3-OHGA (3-hydroxy glutaric acid) accumulate in the brain as well as other organs resulting macrocephaly, brain atrophy and other late-onset clinical presentations.

The present invention provides DNAzyme based approach to knock down the FMR1 mRNA in the embryo and create a robust larval zebrafish model of FXS. Previous studies, as well as data from the present DNAzyme model show that a reduction in FMRP in zebrafish recapitulates molecular and behavioral phenotypes similar to that seen in FXS patients. These include the anatomical features such as long thin dendritic spines, craniofacial abnormalities and bent notochord; molecular phenotypes such as decreased FMR protein and increased mGluR5 protein; and behavioral phenotypes such as increased anxiety, reduced cognition and increased irritability. In the model of the present invention, these behavioral phenotypes are also reversed by the drugs currently prescribed to FXS patients, which strongly validates the DNAzyme based model of FXS.

The already known studies (PLoS One 4(11):e7910, PLoS One 8(3):e51456) however, have made a genetic knockout. In other words, in these studies, the gene is modified to provide altered or absent gene and protein expression. It is a stable, permanent gene deletion which may a) be lethal in certain cases, b) require a significant amount of time and effort to create and maintain. DNAzyme does not change the genome but merely degrades the mRNA which is the premise of the current invention. Such a transient model can be made in a few hours and provides a quick, high-throughput and robust larval model for a number of diseases for which genetic models either do not exist or are not feasible.

### SUMMARY OF THE INVENTION

The present invention provides DNAzymes for reduction in the expression of the target gene implicated in a disease, in zebrafish embryos. The said invention relates to a method of using DNAzymes to create a knockdown in zebrafish embryos.

The invention presents DNAzyme mediated knockdown of fmr1 mRNA, gcdh mRNA and gba1 mRNA thus creating a model of Fragile X syndrome, Glutaricaciduria Type I and Gaucher's Disease in Zebrafish embryos.

In one aspect, the present invention provides a DNAzyme for reducing the expression of target gene in zebrafish embryos comprising using a specific DNAzyme with the core nucleotide sequence of SEQ ID NO. 1 GGCTAGCTACAACGA

In another aspect, the present invention provides a DNAzyme mediated knockdown of Fmr1 mRNA of Genbank Accession number NM_152963 wherein the DNAzyme sequence is selected from a group consisting of SEQ ID NO. 2, 3, 4, 5, 6, 7, 8, 9, 10, 11 and 12 or said DNAzyme targets a sequence that is part of the Fmr1 mRNA sequence of Genbank Accession number NM_152963.

In yet another aspect, the present invention provides a DNAzyme mediated knockdown of gcdh mRNA of Ensmbl Gene ID ENSDARG00000109610 wherein the DNAzyme sequence is selected from a group consisting of SEQ ID NO. 13, 14, 15, 16, 17, 18 and 19 or said DNAzyme targets a sequence that is part of the gcdh mRNA Ensmbl Gene ID ENSDARG00000109610.

In another aspect, the present invention provides a DNAzyme mediated knockdown of gba1 mRNA of Ensmbl gene ID ENSDARG00000076058 wherein the DNAzyme sequence is selected from a group consisting of SEQ ID NO. 20, 21, 22, 23, 24 and 25 or said DNAzyme targets a sequence that is part of the gba1 mRNA of Ensmbl gene ID ENSDARG00000076058. The method of reducing gene expression in a target zebrafish embryo comprising administering one or more DNAzymes into the zebrafish embryo, wherein the DNAzyme comprises a 3′ inverted dT or PEG at the 3′ end is also provided by the invention.

The invention also provides a of reducing gene expression in target comprising administering one or more DNAzymes into the zebrafish embryo wherein the DNAzyme comprises a nucleotide, peptide, protein, chemical linker, or fluorescent tag attached to the 3′ or 5′ end.

In yet another aspect, present invention provides a methodology of using DNAzymes to create a knockdown in a zebrafish embryos of all strains (wild type, inbred strains of all kinds), with the data on Fragile X syndrome, Glutaric aciduria and Gaucher's disease models in Indian wild type zebrafish being presented as examples for such diseases models. Other monogenic diseases models that can be created in zebrafish using this approach, include but not limited to, lysosomal storage disorders, inborn errors of metabolism with a monogenic cause, Marfan's disease, DMD, Autism spectrum disorders with a monogenic cause selected from the group consisting of Fragile X syndrome and Rett's syndrome, Tuberous sclerosis, Neurofibromatosis 1, Cornelia de Lange syndrome, Cohen syndrome, Timothy syndrome, Smith-Lemli-Opitz syndrome, Williams-Beuren syndrome, Dup15q syndromes, Prader-Willi syndrome, Angelman syndrome, 16p11.2 deletion syndrome, Smith-Magenis syndrome, 22q11 duplication syndrome, DeGeorge syndrome (velocardiofacial syndrome) and Phelan-McDermid syndrome.

### BRIEF DESCRIPTION OF THE DRAWINGS

FIG. 1: Strategy for DNAzyme mediated knockdown of target mRNA for model creation in zebrafish.

FIG. 2: A. Sequence of Zebrafish Fmr1 mRNA and DNAzyme target positions; B. In vitro mRNA cleavage assay for DNAzymes; C. Measurement of DNAzyme cleavage efficiency using RT-qPCR; D: Dose and time curves of DNAzyme cleavage of RNA in vitro.

FIG. 3: A. Survival of electroporated zebrafish embryos to show non toxicity of DNAzymes; B. DNAzyme uptake into electroporated zebrafish embryos and their stability in vivo.

FIG. 4: A. Fmr1 mRNA level in DNAzyme treated zebrafish embryos; B. FMRP protein level in DNAzyme treated zebrafish embryos; C. Structural phenotypes observed in DNAzyme treated zebrafish embryos, resembling human FXS phenotypes; D. Behavioral phenotypes observed in DNAzyme treated 7 dpf larvae; E. mGluR5 protein levels in DNAzyme treated zebrafish.

FIG. 5: DNAzymes that cleave the gcdh mRNA identified on the basis of an in-vitro cleavage assay. A. Sequence corresponding to a fragment of the gcdh mRNA, highlighting the target sites for the selected DNAzymes. B. Transcript corresponding to GCDH_test RNA was synthesized in vitro, and incubated with various DNAzymes to measure in vitro cleavage activity. The uncut and cut fragments are indicated. C-D: The amount of gcdh mRNA was quantified by RT-PCR from control and DNAzyme treated embryos at 24, 48 and 72 hpf. The numbers under each lane indicate the fraction of gcdh mRNA remaining at that time point.

FIG. 6: DNAzymes that cleave the gba1 mRNA identified on the basis of an in-vitro cleavage assay. A. Sequence corresponding to a fragment of the gba1 mRNA, highlighting the target sites for the selected DNAzymes. B. Transcript corresponding to a fragment of gba1 mRNA was synthesized in vitro, and incubated with various DNAzymes to measure in vitro cleavage activity. The uncut and cut fragments are indicated.

### DESCRIPTION OF THE INVENTION

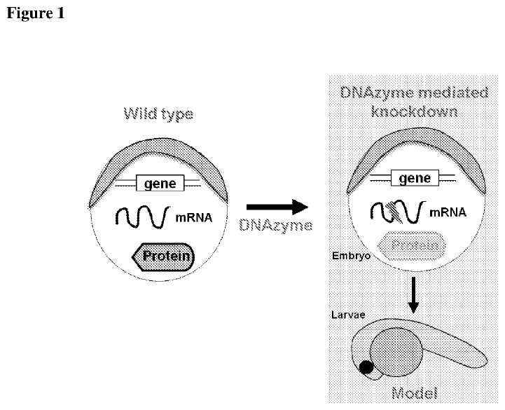

The present invention provides catalytic DNAs (DNAzymes) to knock down the expression of specific genes(s) in the zebrafish embryo, mimicking a natural genetic mutation or deletion found in a disease to create the zebrafish model of that disease (strategy outlined in FIG. 1). Such a model can be used as an inexpensive initial screen to identify drug candidates for repurposing.

A catalytic DNA/DNAzyme is an oligonucleotide that has a catalytic core capable of cleaving a (RNA) phosphodiester bond, flanked by two arms that are complementary to the target RNA of interest. This oligonucleotide on its own is able to cleave the complementary RNA in vitro as well as in vivo. Thus in the present invention, a catalytic DNA is used to knock down the level of an mRNA in a zebrafish embryo, to mimic the diseased state caused due to the loss of function of the corresponding protein.

Therefore in one aspect the present invention provides a specific DNAzyme for reducing the expression of target gene in zebrafish embryos, comprising the following sequence between nucleotide positions 8/9-22/23:

SEQ ID NO. 1:
GGCTAGCTACAACGA

This DNAzyme at positions 1-8 and 22-31 consists of sequences complimentary to any target mRNA. It also has a 3′ inverted dT or other such modifications such as PEG at the 3′ end. A nucleotide, peptide or chemical linker, or fluorescent tag may be attached to the 3′ or 5′ end of the afore-mentioned DNAzyme.

A person skilled in the art will appreciate that the invention provides a method to reduce the expression level of any target mRNA using a specific DNAzyme with the core described in SEQ ID NO. 1, in zebrafish embryos.

More particularly, the present invention provides a DNAzyme mediated knockdown of the zebrafish Fmr1 mRNA of Genbank Accession number NM_152963 comprising a structure selected from the group consisting of:

SEQ ID NO. 2 cdFMRA1:
CTATTTCAGGCTAGCTACAACGAGCAGCGGT
SEQ ID NO. 3 cdFMRA2:
GCTTTCTAGGCTAGCTACAACGATTCACGCA
SEQ ID NO. 4 cdFMRB1:
GCCAGTAAGGCTAGCTACAACGATTAATACA
SEQ ID NO. 5 cdFMRB2:
GTCCATGAGGCTAGCTACAACGAGCCAGTAA
SEQ ID NO. 6 cdFMRB3:
CTCGTCCAGGCTAGCTACAACGAGACGCCAG
SEQ ID NO. 7 cdFMRB6:
TCCTCGCAGGCTAGCTACAACGATTCCACC
SEQ ID NO. 8 cdFMRB7:
AAGCTCCAGGCTAGCTACAACGATCGCTCC
SEQ ID NO. 9 cdFMRC3:
TGACAGTAGGCTAGCTACAACGATCTCATG
SEQ ID NO. 10 cdFMRC4:
AAAGGCCAGGCTAGCTACAACGATGTGACA
SEQ ID NO. 11 cdFMRD2:
TCTGAAAAGGCTAGCTACAACGACGGTTGG
SEQ ID NO. 12 cdFMRE1:
CCACTTCAGGCTAGCTACAACGACGCTCTC

The DNAzyme targets a sequence that is part of the Fmr1 mRNA sequence Genbank Accession number NM_152963. In another embodiment, the present invention also provides a method of reducing Fmr1 expression in a target cell comprising administering one or more DNAzymes.

In yet another embodiment, the present invention provides a strategy for DNAzyme mediated knockdown of the zebrafish gcdh mRNA Ensmbl Gene ID ENSDARG00000109610 comprising a structure selected from the group consisting of:

SEQ ID NO. 13 cdgcd1:
GTGAGTTCAGGCTAGCTACAACGACAGCAGAC
SEQ ID NO. 14 cdgcd2:
TTTGTCATAGGCTAGCTACAACGAGCAGCAG
SEQ ID NO. 15 cdgcd3:
ATCTCCCGAGGCTAGCTACAACGAGGAAATGT
SEQ ID NO. 16 cdgcd4:
CGCTGTCGAGGCTAGCTACAACGACCTCTCCA
SEQ ID NO. 17 cdgcd5:
GATCATGAGGCTAGCTACAACGACTCCTCCT
SEQ ID NO. 18 cdgcd6:
CGTACGCCAGGCTAGCTACAACGAATAACTA
SEQ ID NO. 19 cdgcd7:
GGCATTGAAGGCTAGCTACAACGAGGGGTGCA

The present invention also provides a strategy for DNAzyme mediated knockdown of the zebrafish gba1 mRNA of Ensmbl gene ID ENSDARG00000076058 comprising a structure selected from the group consisting of:

SEQ ID NO. 20 cdGb1:
CCGTCTTTAGGCTAGCTACAACGATGTCAGCT
SEQ ID NO. 21 cdGb2:
GAGCCATGA GGCTAGCTACAACGA CGAAATTC
SEQ ID NO. 22 cdGb3:
TATGTCGCA GGCTAGCTACAACGA TGCACACA
SEQ ID NO. 23 cdGb4:
TATTGCTGA GGCTAGCTACAACGA ATATGATA
SEQ ID NO. 24 cdGb5:
TCAGAGTGA GGCTAGCTACAACGA TCTGAGAG
SEQ ID NO. 25 cdGb6:
GTGCTGGAA GGCTAGCTACAACGA TTCTGACT

The present invention also provides a method of creating a model of Fragile X syndrome and other diseases further exemplified by Gaucher's disease and Glutaric Aciduria Type I in Zebrafish embryos comprising delivering the afore-mentioned DNAzyme(s) into the Zebrafish embryo between 0 hpf (hours post fertilization) to 7 dpf (days post fertilization) to create the knockdown. The DNAzyme is delivered into the Zebrafish embryo by microinjection or electroporation among other methods. A transient reduction in the expression of the target gene is created by introducing sufficient DNAzyme into hundreds of embryos at a time, in order to achieve a significant knockdown without otherwise perturbing the development and maturation of the embryo. As an example, a model for Fragile X syndrome is created using DNAzyme mediated knockdown of Fmr1 mRNA. This leads to the reduction in the protein level of FMR protein (FMRP) and increase in level of mGluR5 protein, characteristic of FXS. The FXS fish display all the molecular and behavioral phenotypes characteristic of FXS patients, and show rescue with drugs prescribed to FXS patients, thus validating the model. It will be appreciated by a person skilled in the art that such a method of creating a model using Zebrafish embryo can be extended to any disease condition not limited by this disclosure.

Thus this platform technology can be extended to make models in zebrafish for a number of monogenetic disorders, pediatric and rare diseases. Data supporting this claim, has been established herein by using the examples of GA1 and Gaucher's disease models.

### Advantages of the Invention

As provided in the present invention, the DNAzyme based models create a transient knockdown of the target mRNA, using a simple oligonucleotide alone, without dependence on any other reagents or the cellular machinery. Therefore, the knockdown is reproducible and robust, and the methodology is quick and inexpensive as compared to the other existing methods. Since the DNAzyme targets the mRNA, there is a direct and immediate effect on the protein level in the embryo, after DNAzyme treatment. However, because of the transient nature of the knockdown, the phenotypes can only be studied in the embryonic and early larval stages (0-7 dpf), which is the window of interest for the study of monogenic rare disease models, and this window is very accessible for study and manipulation in zebrafish.

The present invention provides a robust, inexpensive, quick, high-throughput and simple-to-create models of certain genetic diseases using a DNAzyme based knockdown strategy in Zebrafish. The platform technology can be used to create disease models in Zebrafish, for any disease where a monogenic loss of functionality is the underlying cause. Such a model can be used as an inexpensive initial screen to identify drug candidates for repurposing. The motivation behind the creation of this platform is the unavailability and unaffordability of models, specifically for rare diseases, which was an impediment for drug discovery efforts.

### Examples

1. Several Catalytic DNA Sequences Cleave FMR1 Test RNA Efficiently In Vitro:

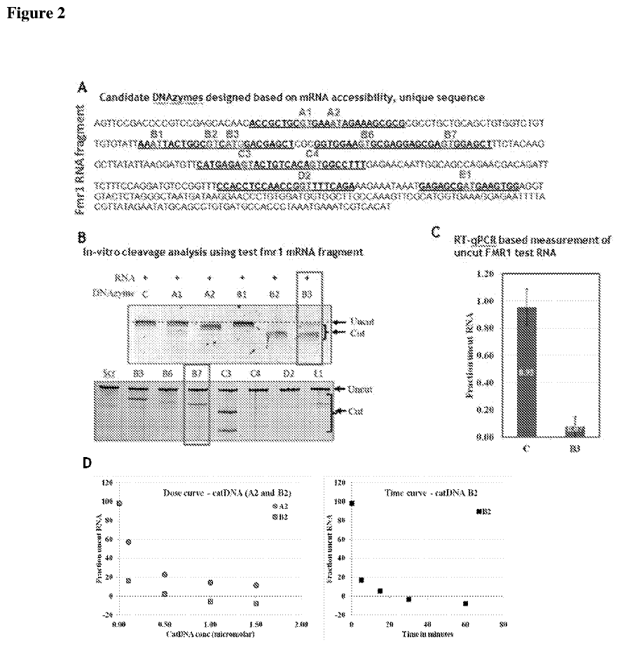

DNA sequence was cloned corresponding to the first 400 nucleotides of the FMR1 mRNA under a T7 promoter in the plasmid pUC57. Using this template, the inventors synthesized the FMR1_400 test RNA by in vitro transcription using the T7 RNA polymerase. Several designed DNAzymes were used in an in vitro cleavage assay using the FMR1_400 test RNA to identify the candidates with the best efficiency. Several DNAzymes such as B3, B2, A2, C3 and B7 were able to cleave the FMR1_400 test RNA efficiently in vitro (FIG. 2). B3 was able to cleave >80% of the full length RNA, within the first five minutes of incubation and was able to do so even at a 1:1 molar ratio (FIG. 2D).

2. Several Catalytic DNA Sequences Cleave GCDH Test RNA Efficiently In Vitro:

DNA sequence corresponding to the first 553 nucleotides of the gcdh mRNA (isoform a) was obtained by reverse transcription-PCR (RT-PCR) from zebrafish total RNA using the primers

(Fwd)
SEQ ID NO. 26
GTAATACGACTCACTATAGGGAACAGCTTTGTCTCGTCTG
(rev)
SEQ ID NO. 27
TCCGTGGTTTGGTTCTGTTAG

A test RNA fragment (GCDH_test RNA) was obtained from this DNA by in vitro transcription using T7 RNA polymerase, and was used as a substrate to test in vitro cleavage activity of the DNAzymes.

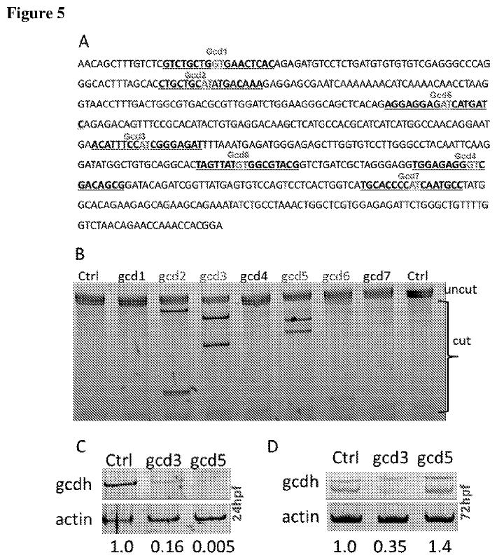

Using an in-silico design approach, the inventors designed seven DNAzyme sequences that target the Zebrafish gcdh mRNA. Target sites were chosen based on the secondary structure of the RNA fragment (as predicted by RNAfold), at regions which were accessible such as part of a bulge, loop or single stranded region (FIG. 5 A). Several designed DNAzymes were used in an in vitro cleavage assay using the GCDH_500 test RNA to identify the candidates with the best efficiency. Several DNAzymes such as Gcd 2, 3, 5 and 6 GCDH_test RNA efficiently in vitro (FIG. 5B). DNAzyme sequences used in the in vitro catalytic activity assay are provided as SEQ ID NO. 13-17 (internal identifiers: gcd1 to gcd7).

3. Several Catalytic DNA Sequences Cleave Gba1 Test RNA Efficiently In Vitro:

DNA sequence was cloned corresponding to the first 500 nucleotides of the gba1 mRNA under a T7 promoter in the plasmid pUC57. Using this template, the inventors synthesized the Gba1_500 test RNA by in vitro transcription using the T7 RNA polymerase.

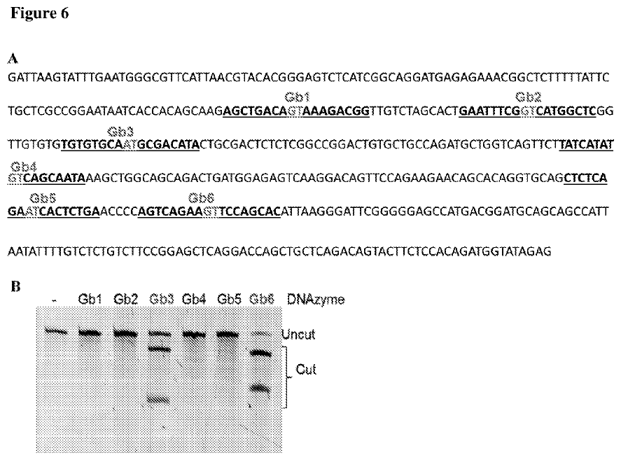

Using an in-silico design approach, the inventors designed six DNAzyme sequences that target the Zebrafish gba1mRNA. Target sites were chosen based on the secondary structure of the RNA fragment (as predicted by RNAfold), at regions which were accessible such as part of a bulge, loop or single stranded region (FIG. 6A). Several designed DNAzymes were used in an in vitro cleavage assay using the Gba1_500 test RNA to identify the candidates with the best efficiency. Several DNAzymes such as Gb3, Gb4 and Gb6 were able to cleave the Gba1_500 test RNA efficiently in vitro (FIG. 6B).

DNAzyme sequences used in the in vitro catalytic activity assay are provided as SEQ ID NO. 18-23 (internal identifiers: gb1 to gb6).

4. DNAzymes is Introduced into Zebrafish Embryos by Electroporation:

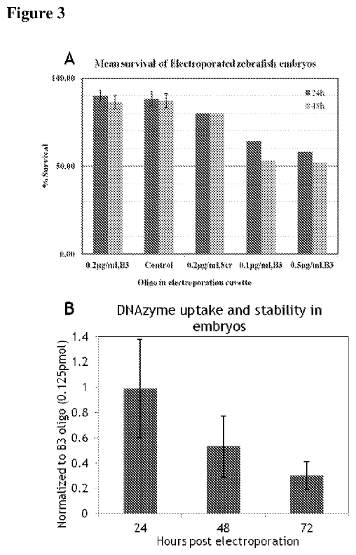

Electroporation was used to efficiently deliver DNAzymes into 0-1 hpf zebrafish embryos. During electroporation, various conditions such as voltage (25-200V), pulse number (2-16), duration (100-200 μsec), DNAzyme concentration (0.1-0.5 μg/μl) and embryo density (25-100 embryos/400p1) were varied to arrive at the optimal set of parameters (50V, 200 μsec, 16 pulses, 0.2 μg/μl DNAzyme, 100 embryos/400 μl) which led to maximal survival of the embryos (FIG. 3A). The amount of catDNA present in the embryo at 24 and 48 hours post treatment was quantified by PCR analysis and found to be comparable to the standard technique of microinjection, with over 80% of input oligonucleotide being delivered into the embryo (FIG. 3B).

5. DNAzymes Result in Significant Knockdown of FMR1 mRNA:

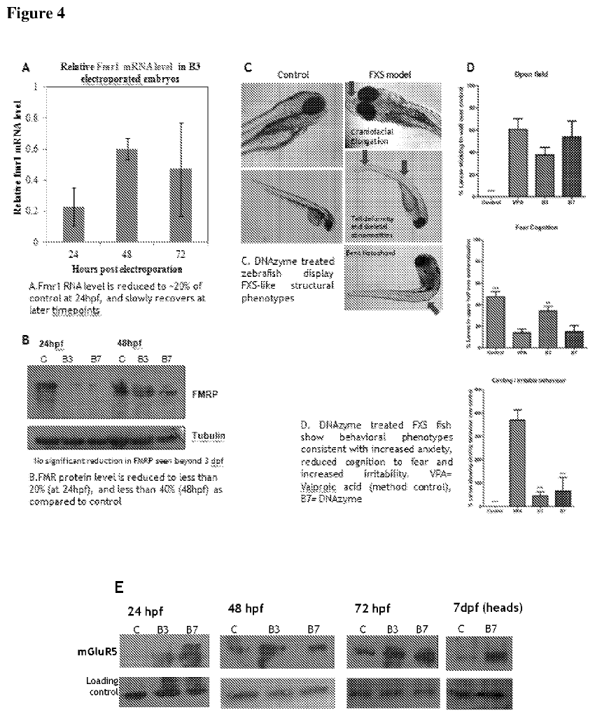

DNAzymes B3, B7 etc. cleave the FMR1_400 test RNA efficiently in vitro, and is expected to do the same with the FMR1 mRNA in vivo. In order to test this, FMR1 mRNA level was measured by using semi-quantitative RT-PCR assay, from DNAzyme treated and control embryos (which were treated with an oligo that has a scrambled sequence), at various timepoints. Briefly different sets of zebrafish embryos were electroporated in the presence of DNAzyme B3/B7 at the 1-2 cell stage. At 24 hrs, 48 hrs or 72 hours post treatment, embryos were collected, pooled in batches of 5 and total RNA was extracted by standard methods. From this RNA, the level of FMR1 mRNA was measured and quantified and found to be significantly reduced in the DNAzyme treated embryos relative to control. The FMR1 level was normalized to an internal control (act1 mRNA) and is presented as a chart, in treated embryos relative to control (FIG. 4A).

6. DNAzymes Result in Significant Knockdown of Gcdh mRNA:

DNAzymes Gcd2,3 and 5 cleave the GCDH_test RNA efficiently in vitro, and are expected to do the same with the gcdh mRNA in vivo. In order to test this, DNAzymes were introduced by electroporation as described previously for the FMR1 DNAzymes. Subsequently, gcdh mRNA level was measured by using semi-quantitative RT-PCR assay, from DNAzyme treated and control embryos at various timepoints. Briefly different sets of zebrafish embryos were electroporated in the presence of DNAzyme oligo at the 1-2 cell stage. At 24 hrs, 48 hrs or 72 hours post treatment, embryos were collected, pooled in batches of 50 and total RNA was extracted by standard methods. From this RNA, the level of gcdh mRNA was measured by RT-PCR, electrophoresed on native PAGE and quantified using ImageJ. The gcdhmRNA level was normalized to an internal control (act1 mRNA) for each sample, and then normalized to control embryos. The fraction of gcdh mRNA remaining, in treated embryos relative to control, is indicated below the corresponding lane in FIG. 5C, D. The level of gcdh mRNA was found to be significantly reduced in the DNAzyme treated embryos relative to control, for both gcd3 and gcd5 at 24 hpf, and for as long as 72 hpf in the case of gcd5.

7. DNAzymes Result in Significant Knockdown of FMR Protein and the Consequent Molecular and Behavioral Phenotypes:

Similarly, FMRP levels were measured in untreated and treated embryos by using whole-embryo Western analysis. A similar experimental protocol was followed for DNAzyme treatment, embryos were collected at various time points post treatment, pooled in batches of 5, and total protein was extracted. FMRP level was measured by standard SDS-PAGE followed by Western Blotting using a zebrafish FMRP antibody (FIG. 4B), and found to be significantly reduced. Anatomical phenotypes such as craniofacial elongation, bent notochord and tail deformities were observed in the DNAzyme treated zebrafish at the 48-72 hpf stages (FIG. 4C).

Behavioral assays were carried out at the 7 dpf stage to measure anxiety (open-field test), fear cognition (red-bar test) and irritability (circling behavior). VPA, an agent known to induce symptoms of Autism spectrum disorders in mice models (Wagner et al. 2006), was used as a positive control in these assays. The DNAzyme treated FXS fish show increased anxiety, decreased fear cognition and increased irritability (like VPA) compared to control zebrafish larvae (FIG. 4D).

In the DNAzyme model of FXS, the knock down of FMRP using DNAzyme technology, results in lasting changes in downstream targets (mGluR5) and is presented in the Western blot shown in FIG. 4E. This indicates that the behavioral phenotypes observed in these DNAzyme treated larvae at 7 dpf are due to the DNAzyme mediated FMRP knockdown and the consequent increase in mGluR5 among other changes.

We claim:

1. A method of developing a model of Fragile X syndrome in zebrafish embryos comprising delivering one or more DNAzyme sequence into the zebrafish embryo at 0 hpf (hours post fertilization) to 7 dpf (days post fertilization) to knockdown FMR1 mRNA expression.

2.-3. (canceled)

4. The method of claim 1 wherein the DNAzyme sequence is selected from a group consisting of SEQ ID NO. 2, 3, 4, 5, 6, 7, 8, 9, 10, 11, or 12, and targeting any sequence that is part of the Fmr1 mRNA sequence of Genbank Accession number NM 152963.

5.-7. (canceled)

8. A method of developing a model of Glutaric Aciduria Type 1 (GA1) in zebrafish embryos comprising delivering one or more DNAzyme sequence into the zebrafish embryo at 0 hpf (hours post fertilization) to 7 dpf (days post fertilization) to knockdown gcdh mRNA expression.

9.-10. (canceled)

11. The method of claim 8, wherein the DNAzyme sequence is selected from a group consisting of SEQ ID NO. 13, 14, 15, 16, 17, 18. or 19 and targeting any sequence that is part of the gcdh mRNA Ensmbl Gene ID ENSDARG00000109610.

12.-14. (canceled)

15. A method of developing a model for Gaucher's Disease in zebrafish embryos comprising delivering one or more DNAzymes sequence into the zebrafish embryo at 0 hpf (hours post fertilization) to 7 dpf (days post fertilization) to knockdown gba1 mRNA expression.

16.-17. (canceled)

18. The method of claim 15 wherein the DNAzyme sequence is selected from a group consisting of SEQ ID NO. 20, 21, 22, 23, 24, or 25 and targeting a sequence that is part of the gba1 mRNA of Ensmbl gene ID ENSDARG00000076058.

19.-21. (canceled)

22. A method to reduce the expression level of a target mRNA in zebrafish embryos, wherein the said method comprises administering a DNAzyme sequence comprising the core nucleotide sequence of SEQ ID NO. 1 (GGCTAGCTACAACGA) to a zebrafish embryo.

23. A method of developing a model of monogenic diseases in zebrafish embryos comprising delivering a DNAzyme sequence targeting the causal gene, into the zebrafish embryo between 0 hpf (hours post fertilization) to 7 dpf (days post fertilization) resulting in knocking down of the causal gene.

24. (canceled)

25. The method of claim 23, wherein the DNAzyme sequence targets a mRNA that is specific to a disease condition.


SEQUENCE LISTING (27 sequences)

SEQ ID NO: 1 - DNA, 15 bp, Zebrafish
ggctagctacaacga

SEQ ID NO: 2 - DNA, 31 bp, Zebrafish
ctatttcaggctagctacaacgagcagcggt

SEQ ID NO: 3 - DNA, 31 bp, Zebrafish
gctttctaggctagctacaacgattcacgca

SEQ ID NO: 4 - DNA, 31 bp, Zebrafish
gccagtaaggctagctacaacgattaataca

SEQ ID NO: 5 - DNA, 31 bp, Zebrafish
gtccatgaggctagctacaacgagccagtaa

SEQ ID NO: 6 - DNA, 31 bp, Zebrafish
ctcgtccaggctagctacaacgagacgccag

SEQ ID NO: 7 - DNA, 30 bp, Zebrafish
tcctcgcaggctagctacaacgattccacc

SEQ ID NO: 8 - DNA, 30 bp, Zebrafish
aagctccaggctagctacaacgatcgctcc

SEQ ID NO: 9 - DNA, 30 bp, Zebrafish
tgacagtaggctagctacaacgatctcatg

SEQ ID NO: 10 - DNA, 30 bp, Zebrafish
aaaggccaggctagctacaacgatgtgaca

SEQ ID NO: 11 - DNA, 30 bp, Zebrafish
tctgaaaaggctagctacaacgacggttgg

SEQ ID NO: 12 - DNA, 30 bp, Zebrafish
ccacttcaggctagctacaacgacgctctc

SEQ ID NO: 13 - DNA, 32 bp, Zebrafish
gtgagttcaggctagctacaacgacagcagac

SEQ ID NO: 14 - DNA, 31 bp, Zebrafish
tttgtcataggctagctacaacgagcagcag

SEQ ID NO: 15 - DNA, 32 bp, Zebrafish
atctcccgaggctagctacaacgaggaaatgt

SEQ ID NO: 16 - DNA, 32 bp, Zebrafish
cgctgtcgaggctagctacaacgacctctcca

SEQ ID NO: 17 - DNA, 31 bp, Zebrafish
gatcatgaggctagctacaacgactcctcct

SEQ ID NO: 18 - DNA, 31 bp, Zebrafish
cgtacgccaggctagctacaacgaataacta

SEQ ID NO: 19 - DNA, 32 bp, Zebrafish
ggcattgaaggctagctacaacgaggggtgca

SEQ ID NO: 20 - DNA, 32 bp, Zebrafish
ccgtctttaggctagctacaacgatgtcagct

SEQ ID NO: 21 - DNA, 32 bp, Zebrafish
gagccatgaggctagctacaacgacgaaattc

SEQ ID NO: 22 - DNA, 32 bp, Zebrafish
tatgtcgcaggctagctacaacgatgcacaca

SEQ ID NO: 23 - DNA, 32 bp, Zebrafish
tattgctgaggctagctacaacgaatatgata

SEQ ID NO: 24 - DNA, 32 bp, Zebrafish
tcagagtgaggctagctacaacgatctgagag

SEQ ID NO: 25 - DNA, 32 bp, Zebrafish
gtgctggaaggctagctacaacgattctgact

SEQ ID NO: 26 - DNA, 40 bp, Zebrafish
gtaatacgactcactatagggaacagctttgtctcgtctg

SEQ ID NO: 27 - DNA, 21 bp, Zebrafish
tccgtggtttggttctgttag